(19) United States
(12) Patent Application Publication
(10) Pub. No.: US 2022/0349007 A1
(43) Pub. Date: Nov. 3, 2022

(54) Method of Culturing and Analyzing at Least One Cell in a Microchamber Configured to Allow for Optical Inspection of the at Least One Cell, a Device for Use in the Method, a System and a Computer Program for Performing One or More of the Steps of the Method

(71) Applicant: BICO Group AB, Göteborg (SE)

(72) Inventors: Erik Gatenholm, Göteborg (SE); Hector Martinez, Göteborg (SE); Jonas Schöndube, Freiburg (DE)

(21) Appl. No.: 17/732,962

(22) Filed: Apr. 29, 2022

(30) Foreign Application Priority Data
Apr. 29, 2021 (SE) 2150546-6

(51) Int. Cl. C12Q 1/6881; C12M 3/06; C12M 1/00; B01L 3/00; G01N 21/03

(52) U.S. Cl. CPC C12Q 1/6881; C12M 23/16; C12M 23/28; B01L 3/502761; G01N 21/03; B01L 2200/0647; B01L 2200/10; B01L 2400/0436; C12Q 1/6869

(57) ABSTRACT

The disclosure relates to a method of culturing and analyzing at least one cell in a microchamber configured to allow for optical inspection of the at least one cell, wherein liquid is extracted from the microchamber for analysis, characterized in that the analysis returns information about particles secreted from the at least one cell and that this information can be correlated to the individual cell and/or cell population. The disclosure further relates to a device for use in the method and a system and a computer program for performing one or more of the steps of the method.

### CROSS-REFERENCE TO RELATED APPLICATION

This application claims the benefit of priority under 35 U.S.C. § 119 of Swedish patent application number 2150546-6 filed Apr. 29, 2021, which is hereby incorporated by reference in its entirety.

### TECHNICAL FIELD

The present disclosure relates to a method of culturing and analyzing at least one cell in a microchamber configured to allow for optical inspection of the at least one cell, a device for use in the method, a system for performing one or more of the steps of the method, and a computer program for use in the method.

### BACKGROUND ART

Cai W. et al. (Lab Chip, 2018, 18, 3154-3162) discloses a single-cell translocation and secretion assay. Contreras-Naranjo et al. (Lab Chip, 2017, 17, 3358-3577), WO2020/249130 and WO2019/145433 represent other pieces of prior art within the field.

WO2017/027549 discloses magnetic single cell arrays of probing cell-drug and cell-cell communication. WO2020/047071, US2019/0285618, US2018/0299362 and US2018/066307 represent other pieces of prior art within the field.

There is a need for improved methods and devices for culturing individual cells and assigning genotypic and/or phenotypic features to the individual cells.

### SUMMARY

It is an object of the present disclosure to mitigate, alleviate or eliminate one or more of the above-identified deficiencies and disadvantages in the prior art and solve at least the above mentioned problem. According to a first aspect there is provided a method of culturing and analyzing at least one cell in a microchamber configured to allow for optical inspection of the at least one cell, wherein liquid is extracted from the microchamber for analysis, characterized in that the analysis returns information about particles secreted from the at least one cell and that this information can be correlated to the individual cell and/or cell population.

Hereby, by extracting and analyzing the particles secreted from an individual cell, information obtained by the analysis can be assigned to the individual cell and allows for determining features of the cell, such as its suitability and applicability for various uses, such as for gene therapy, cell therapy, stem cell therapy, regenerative medicine, bioprinting and/or biofabrication of human tissues and organs for implantation.

According to some embodiments, the liquid extraction is performed by means of a microfluidic valve and channel or a micropipette.

Hereby, the secreted particles can be separated from the cell(s) that the particles has/have been secreted from.

According to some embodiments, the particles secreted from the at least one cell are extracellular vesicles, such as exosomes.

It has been shown, that exosomes are useful for analyses like single-cell omics and live single-cell RNA sequencing.

According to some embodiments, the particles secreted from the at least one cell are separated from the cells by any one of the following alternatives:

(1) By means of a disposable microfluidic device, the method comprises a microfluidic channel that is thinner than a normal cell, and a particle collection well, wherein the microfluidic channel is used for transporting particles from a cell culturing well to the particle collection well, where the particles can be taken for further analysis.

(2) By (i) adding micromagnetic particles having the ability to bind to the particles secreted from the at least one cell to a cell culturing well; (ii) aspirating the at least one cell, particles secreted from the at least one cell, and the micromagnetic particles into an acoustic trap; (iii) separating the secreted particles, which bind to the magnetic microparticles via physical and/or chemical binding, from the at least one cell; and (iv) dispensing the secreted particles into a particle collection well, where the particles can be taken for further analysis.

In accordance with alternative (2), in step (iii), the acoustic trap is deactivated, whereby the magnetic microparticles, having secreted particles bound to them, and the cells are released to at least one source well, in which the magnetic microparticles and the secreted particles are immobilized by applying a magnetic field. Further, the cells can be dispensed separately from the at least one source well by applying a positive pressure within the source well. Still further, the secreted particles can be released from the magnetic microparticles by applying a physical, chemical or enzymatic reaction, and thereafter being dispensed from the source well.

(3) By separating the at least one cell from the particles secreted from the at least one cell using electromechanical principles on a microfluidic chip, so that the secreted particles are collected in a particle collection well, where the particles can be taken for further analysis.

These alternatives provide concrete ways of separating and enriching the secreted particles from the cell(s), which is an essential step of a live single cell RNA sequencing workflow.

According to some embodiments, the particles secreted from the at least one cell are separated and/or cleaned up by means of lateral displacement, SAW, acoustic technology, coated pillers or beads, electrophoresis or ultracentrifugation.

A plurality of technologies, combined or used separately, can be used for the purposes of the present disclosure.

According to some embodiments, the analysis returning information about particles secreted from the at least one cell includes one or more of the following alternatives: single-cell omics, live single-cell RNA sequencing, single-cell identification at genotypic and/or phenotypic level, proteomics, mass spectrometry.

Hence, information obtained by analyzing the secreted particles, such as exosomes, can provide useful information about the cell to which the secreted particles can be assigned.

According to some embodiments, the method can be used for identifying cells that can be used for various uses, such as for gene therapy, cell therapy, stem cell therapy, regenerative medicine, bioprinting and/or biofabrication of human tissues and organs for implantation.

Hereby, the individual cell(s) can be used for a plurality of applications and purposes, depending on the analyses of its secreted particles.

According to some embodiments, one or more presteps are performed, wherein single cells, such as at least one cell, are provided, sorted and stored under conditions allowing intracellular particles, such as exosomes, to be secreted from the single cells.

Hereby, the method also involves steps that are necessary for providing the cells to be used in the later steps of the method.

According to some embodiments, the analysis of particles secreted and separated from the at least one cell involves the steps of

(i) opening up the particle, to provide access to the RNA content of the particle;

(ii) reverse transcribing the RNA content, to convert the RNA content into at least one DNA strand;

(iii) amplifying the DNA strand; and

(iv) sequencing the DNA strands, to provide genetic information that can be correlated to the individual cell.

Hereby, the method also involves steps that are useful for the analysis and for providing the genetic information that can be correlated to the individual cell and/or cell population.

According to a second aspect there is provided a device for use in culturing and analyzing at least one cell, comprising a microchamber configured to allow for optical inspection of the at least one cell, to be used in the method of the first aspect.

According to some embodiments, the device is a disposable microfluidic device.

According to some embodiments, the device is a microchamber for culturing individual cells, which microchamber allows for live cell imaging.

According to some embodiments, the device allows for extraction and/or analysis of particles secreted from the at least one cell.

According to some embodiments, the analysis of particles secreted from the at least one cell can be assigned to the individual cell and/or cell population.

According to some embodiments, the device is implemented on a chip comprising a plurality of microchambers and/or microfluidic devices.

According to a third aspect there is provided a system comprising at least one device for use in culturing and analysis of at least one cell, which device comprises a microchamber configured to allow for optical inspection of the at least one cell, and at least one control unit configured to allow for extraction of liquid from the microchamber for analysis, wherein the at least one control unit can be integrated into the device or being a separate unit, for performing one or more of the steps of the method of the first aspect.

Such system can include all aspects of the methods of the disclosure, thereby combining, in an automated manner, different technologies, for an efficient and qualitative use for the end-user.

According to some embodiments, the system further comprises at least one dispensing device, configured to allow for dispensing of particles secreted from the at least one cell into a particle collection well for further analysis of the secreted particles.

According to some embodiments, the system further comprises at least one of the following components: means for cell sorting, means for multiomics analysis, means for reverse transcription, means for amplification and means for sequencing.

Hereby, the system includes several independent devices and/or apparatuses, and/or devices and/or apparatuses that are integrated to perform tasks of several components related to the method of the first aspect.

According to a fourth aspect there is provided a computer program for use in culturing and analysis of at least one cell in a microchamber, comprising instructions which, when executed on at least one processor, cause the at least one processor to carry out the method of the first aspect.

Hereby, one or several steps of the method can be integrated and run via a single computer program, thereby enabling a smooth and efficient use and performance of the method of the first aspect.

Effects and features of the second, third and fourth aspects are to a large extent analogous to those described above in connection with the first aspect. Embodiments mentioned in relation to the first aspect are largely compatible with the second, third and fourth aspects.

The present disclosure will become apparent from the detailed description given below. The detailed description and specific examples disclose preferred embodiments of the disclosure by way of illustration only. Those skilled in the art understand from guidance in the detailed description that changes and modifications may be made within the scope of the disclosure.

Hence, it is to be understood that the herein disclosed disclosure is not limited to the particular component parts of the device described or steps of the methods described since such device and method may vary. It is also to be understood that the terminology used herein is for purpose of describing particular embodiments only, and is not intended to be limiting. It should be noted that, as used in the specification and the appended claim, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the words “comprising”, “including”, “containing” and similar wordings does not exclude other elements or steps.

### Terminology

The term “microchamber” is to be interpreted as any chamber allowing for culturing of individual cells, such as wells on a reaction plate or microtiter plate.

The term “acoustic trapping” refers to the concept of immobilizing particles and cells in the node of an ultrasonic standing wave field. By using a localized acoustic field it is thereby possible to capture and hold cells and particles against a flow, in order to enrich and/or isolate biological particles in a liquid.

The term “I-DOT” refers to a technology platform used for dispensing droplets containing small molecules, particles and single cells in a controlled manner. See “https://dispendix.com/idot-non-contact-liquid-handler/” for reference.

The term “extracellular vesicles” refers to lipid bound vesicles secreted by cells into the extracellular space. The three main subtypes of EVs are microvesicles (MVs), exosomes, and apoptotic bodies, which are differentiated based upon their biogenesis, release pathways, size, content, and function. The content, or cargo, of EVs consists of lipids, nucleic acids, and proteins. In this disclosure, “secreted particles” typically refer to “extracellular vesicles”. The following article (“Biological properties of extracellular vesicles and their physiological functions”) is incorporated herein as a reference: https://www.ncbi.nlm.nih.gov/pmc/articles/PMC4433489/.

The term “micromagnetic particles” refers to particles used for biological assays and methods for separating biomolecule and cells by way of magnetic principles. The skilled person would be aware of suitable particles depending on context and application.

### BRIEF DESCRIPTIONS OF THE DRAWINGS

The above objects, as well as additional objects, features and advantages of the present disclosure, will be more fully appreciated by reference to the following illustrative and non-limiting detailed description of example embodiments of the present disclosure, when taken in conjunction with the accompanying drawings.

FIG. 1-10 shows some embodiments of the present disclosure, the embodiments being related to alternative technologies of a live single-cell RNA sequencing workflow (LscRNA-Seq): (1) Disposable microfluidic chip with a plurality, such as hundreds/thousands of wells for collection of particles secreted from cells, such as extracellular vesicles, particularly exosomes, (2) sorting particles secreted from cells using acoustic trapping+single cell/small volume dispensing, such as i-DOT technology, and integrating these technologies for lscRNA-Seq, and (3) sorting particles secreted from cells using microfluidic chip based on electromechanical principles, and integrating this technology for lscRNA-Seq,

### DETAILED DESCRIPTION

The present disclosure will now be described in further detail. The disclosure may, however, be embodied in other forms and should not be construed as limited to the herein disclosed embodiments. The disclosed embodiments are provided to fully convey the scope of the disclosure to the skilled person.

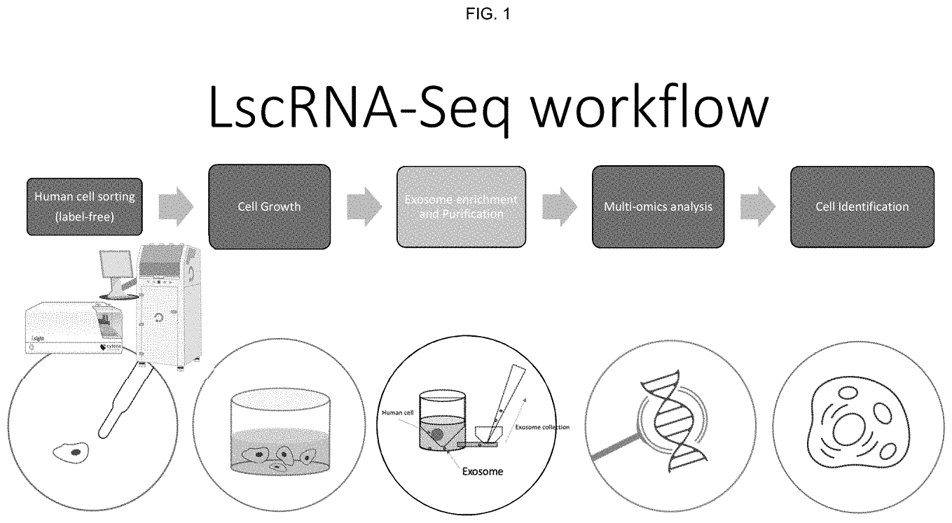

Turning to FIG. 1, disclosing a lscRNA-Seq workflow (Lsc=live single cell), the following steps are typically part of the workflow:
(i) Human cell sorting (label-free), wherein cell sorting is performed directly in a microfluidic chip with assurance of colonality.
(ii) Cell Growth, wherein cells proliferate in individual wells (under standard culture conditions). Particles, such as exosomes, are released (secreted) from the cells and act as mediators of cell-to-cell communication.
(iii) Exosome enrichment and purification, wherein exosomes secreted by the cells are collected, enriched and purified within or outside the chip with various techniques.
(iv) Multi-omics analysis, wherein purified exosomes are prepared for scRNA-Seq to analyze the cell genotype. Multi-omics analysis is also possible.
(v) Cell Identification, based on multi-omics analysis, wherein cells from individual wells are identified at the genotypic and phenotypic level.

In one embodiment, a system for lscRNA-Seq (live, single cell RNA sequencing) according to the present disclosure could be built up as follows:

A system for isolation of particles secreted from cells, such as exosomes. The system is using chips with micro fluidic channels and wells where single cells are sorted into and stored. These cells are then producing these exosomes under normal in-vitro conditions. The exosomes are being channeled and travels through the micro fluidic channels to collection wells where the system collects them, inserts a bead (barcodes and enzymes) into the exosome. Or breaks the exosome apart and introduces it to a bead. Then once the exosome is opened up, reversed transcription occurs and the RNA is converted into DNA strands. Then these DNA strands will be amplified in a regular PCR and then you can run NGS (next generation sequencing) on it. This system would be called a “live, single cell RNA Sequencing” (lscRNA Seq).

Any cells of interest to be analysed for genotypic and/or phenotypic traits could be used in accordance with the present invention.

In the context of the present disclosure, three alternatives for combining multiple technologies to isolate, enrich and purify particles secreted from cells, such as extracellular vesicles, particularly exosomes, as a means to do single cell identification are disclosed: (1) A disposable microfluidic chip with hundreds/thousands of wells for collection of particles, such as exosomes. (2) Sorting particles, such as exosomes, using acoustic trapping, combined with small particle dispensing (such as I-DOT). These technologies are integrated for LscRNA-Seq. (3) Sorting particles (such as exosomes) using a microfluidic chip based on electromechanical principles, and integrating this technology for LscRNA-Seq.

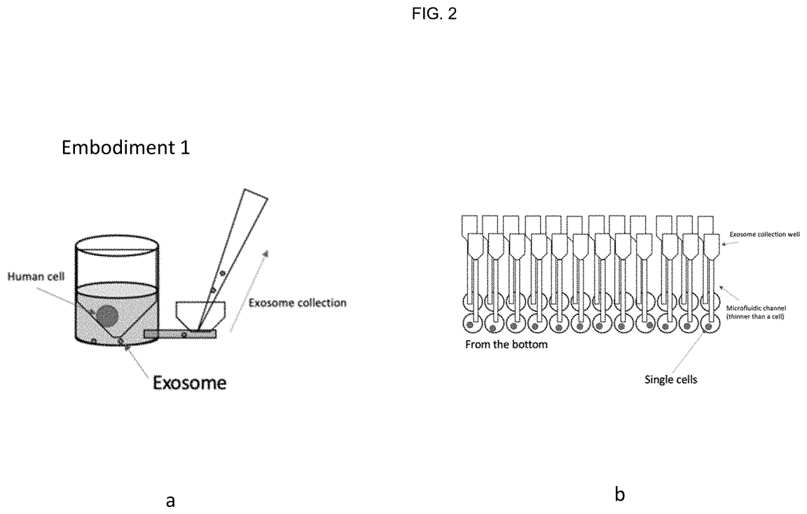

In accordance with FIG. 2 (a, b), a first embodiment of the method of the present disclosure is described, related to a device comprising a disposable microfluidic chip with a plurality (such as hundreds or thousands) of wells for collection of particles secreted from cells, such as exosomes. The device contains small 100 micron-1 mm wells connected to a microfluidic channel (preferably between 20 to 250 nm in diameter and up to 1 micrometer), leading to a particle (exosome) collection well.

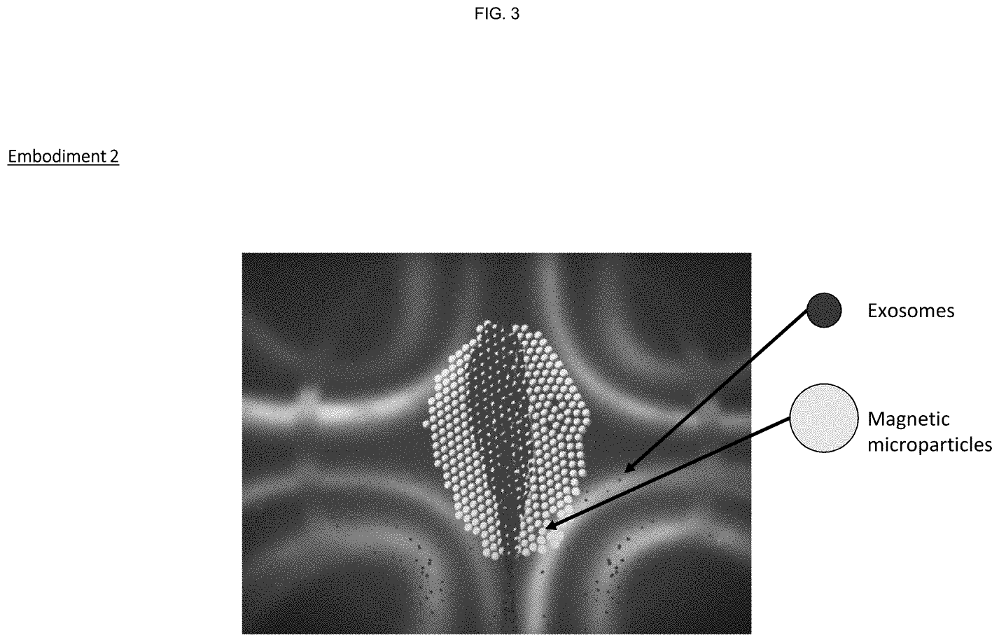

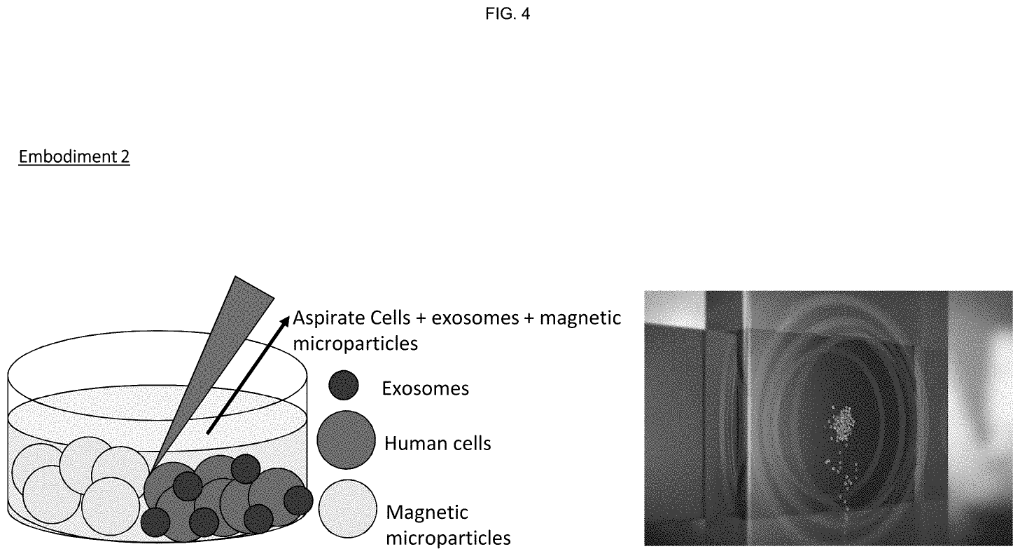

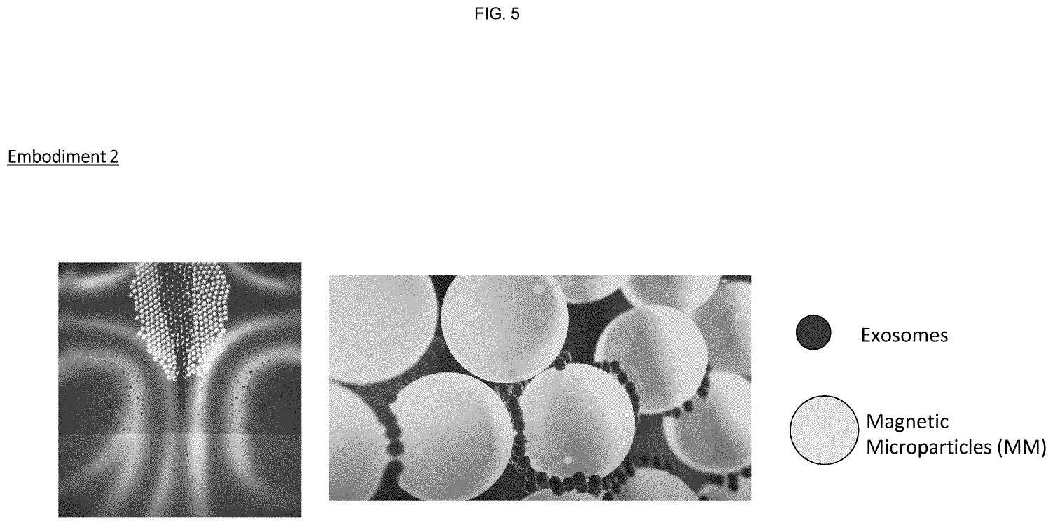

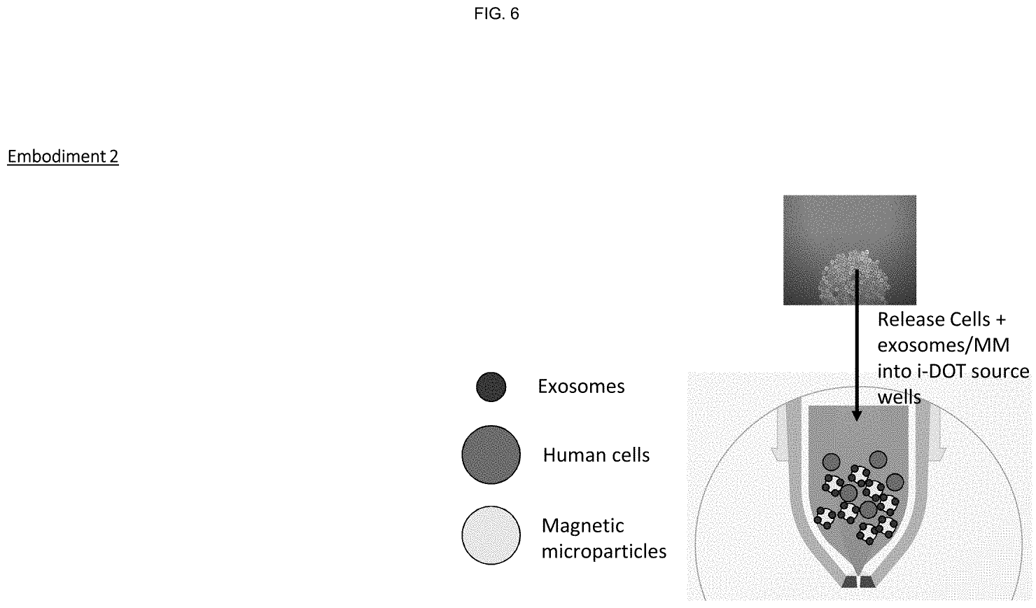

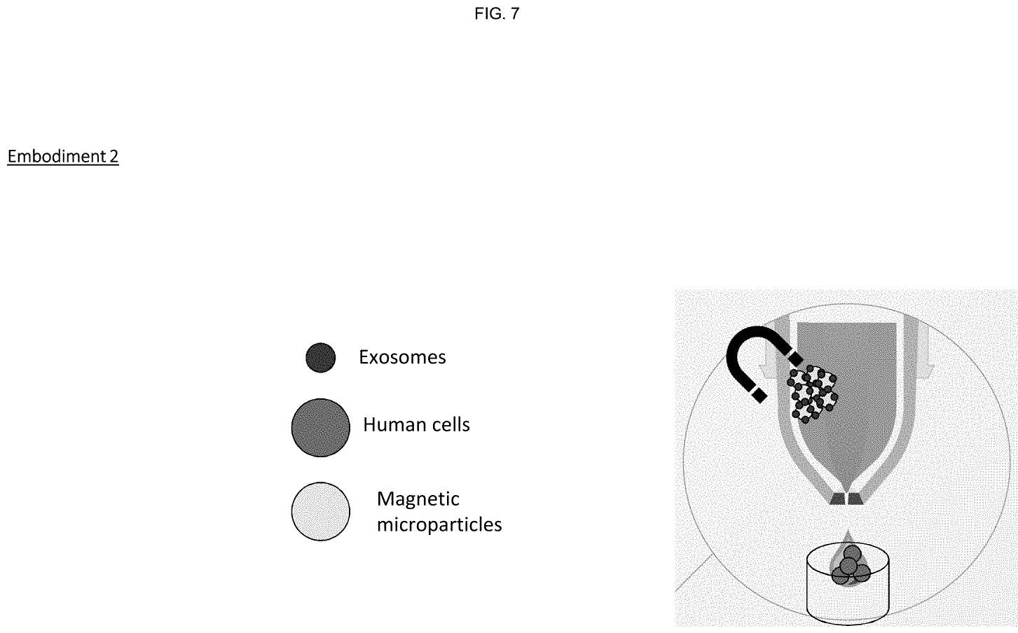

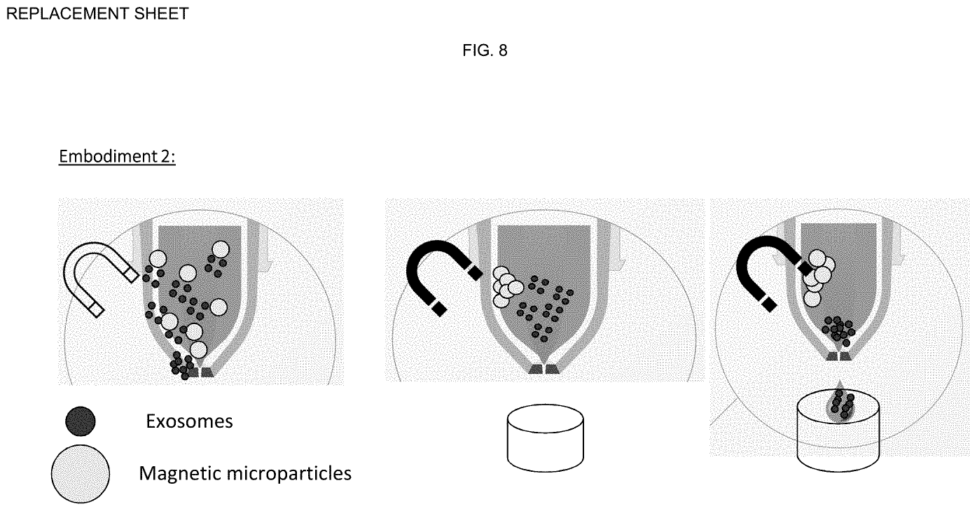

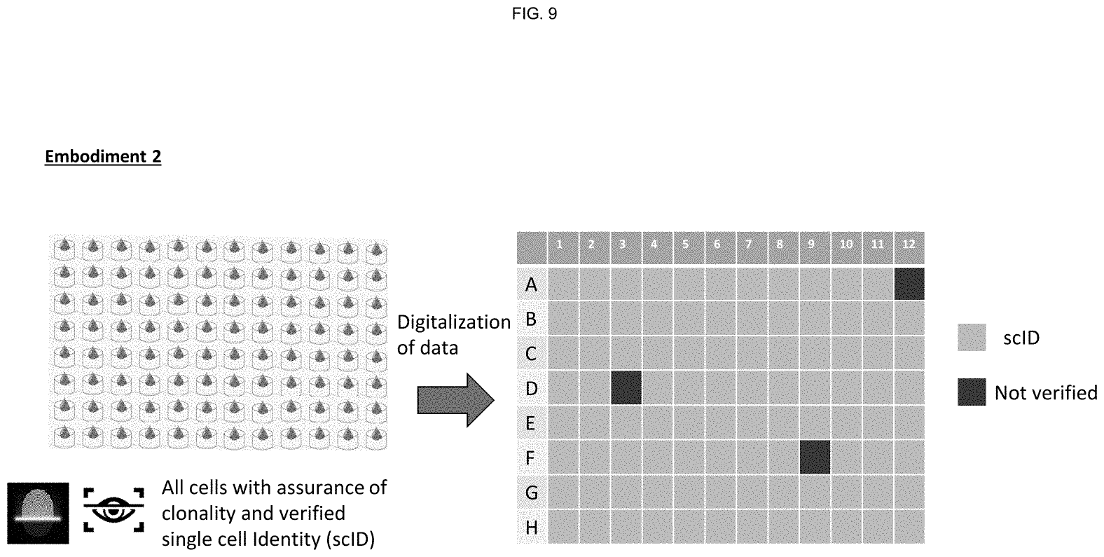

In accordance with FIG. 3-10, a second embodiment of the method of the present disclosure is described, related to the sorting of particles secreted from a cell (such as exosomes) using acoustic trapping combined with small particle dispensing (such as I-DOT), whereby these technologies are integrated for LscRNA-Seq. FIG. 3 shows exosomes and magnetic microparticles in an acoustic trap. To start with, magnetic microparticles are added to each well. Thereafter (FIG. 4), cells and particle (exosome) suspension and magnetic microparticles are aspirated into the acoustic trap (while the transducer is ON) to start the particle (exosome) isolation. The glass capillary used can be washed between wells to remove any cell material. Next (FIG. 5), as particles (exosomes) are aspirated into the acoustic trap, they bind to the magnetic microparticles (MM) for enrichment and isolation from larger volumes. The interaction can be a physical or chemical binding (stable/unstable, weak/strong interaction). Thereafter (FIG. 6), cells and particles (exosomes)/magnetic microparticles (MM) are released from the acoustic trap (by turning off the electric field) and into dispensing wells (such as i-DOT source wells) for further processing. Subsequently (FIG. 7), particles (exosomes)/magnetic microparticles (MM) are held in place in the dispensing wells (i-DOT wells) with the help of a magnetic field. Cells are dispensed from the dispensing (i-DOT) well by applying a positive pressure and are collected in a microfluidic chip or any sterile substrate for further processing. At this step, cells are stored in standard cell culture conditions until multi-omics analysis is performed to identify each cell at the genotypic and phenotypic level. Thereafter (FIG. 8), particles (exosomes) are released from the magnetic microparticles by a physical, chemical or enzymatic reaction. The MMs are held in place by a magnetic field. Finally, the particles (exosomes) are dispensed from the dispensing (i-DOT) well and are collected in a microfluidic chip, any sterile substrate or PCR plate or other suitable substrate, for multi-omics analysis to identify each cell at the genotypic and phenotypic level. (FIG. 9) Once multi-omics analysis has been run and each cell has been identified at the genotypic and phenotypic level, this data is digitalized and the automated LscRNA-Seq platform shows the user which cells and cell populations have the genotypic and phenotypic composition of interest and thus can be used for the target application. The platform will automatically dispose the cells which are outside the pre-defined boundaries.

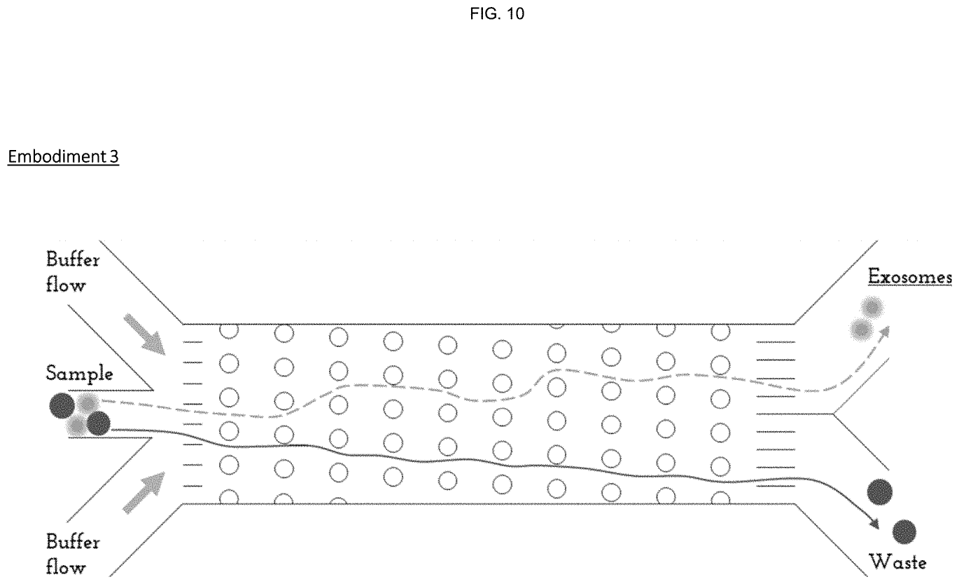

In accordance with FIG. 10, a third embodiment of the method of the present disclosure is described, related to the sorting of particles secreted from cells (such as exosomes) using a microfluidic chip based on electromechanical principles, and integration of this technology for LscRNA-Seq. A sample containing particles secreted from a cell (such as exosomes) and other secretion products (waste) is transported through the chip by a buffer flow, and separation of particles (exosomes) from waste is conducted by means of electromechanical principles applied in the microfluidic chip. This embodiment is based on the combination of deterministic lateral displacement (DLD) and dielectrophoretic forces (DEP), which are exerted by the three-dimensional electrodes in the device.

Regarding conditions to allow for particles, such as exosomes, to be secreted from the at least one cell and conditions and methods to apply in order to open up particles, such as extracellular vesicles, particularly exosomes, further technical details can be found in these references, which hereby are incorporated as references: “https://www.systembio.com/smartsec-single-for-ev-isolation” and “https://www.systembio.com/services/exosome-services/exo-ngs”.

Thus, the first aspect of this disclosure shows a method of culturing and analyzing at least one cell in a microchamber configured to allow for optical inspection of the at least one cell, wherein liquid is extracted from the microchamber for analysis, characterized in that the analysis returns information about particles secreted from the at least one cell and that this information can be correlated to the individual cell and/or cell population.

Embodiments of the first aspect include that:
the liquid extraction is performed by means of a microfluidic valve and channel or a micropipette.
the particles secreted from the at least one cell are extracellular vesicles, such as exosomes.
the particles secreted from the at least one cell are separated from the cells by any one of the following alternatives:
   (1) by means of a disposable microfluidic device, the method comprises a microfluidic channel that is thinner than a normal cell, and a particle collection well, wherein the microfluidic channel is used for transporting particles from a cell culturing well to the particle collection well, where the particles can be taken for further analysis;
   (2) by adding micromagnetic particles having the ability to bind to the particles secreted from the at least one cell to a cell culturing well, and aspirating the at least one cell, particles secreted from the at least one cell, and the micromagnetic particles into an acoustic trap, and separating the secreted particles, which bind to the magnetic microparticles via physical and/or chemical binding, from the at least one cell, and dispensing the secreted particles into a particle collection well, where the particles can be taken for further analysis; and
   (3) by separating the at least one cell from the particles secreted from the at least one cell using electromechanical principles on a microfluidic chip, so that the secreted particles are collected in a particle collection well, where the particles can be taken for further analysis.
the particles secreted from the at least one cell are separated and/or cleaned up by means of lateral displacement, SAW, acoustic technology, coated pillers or beads, electrophoresis or ultracentrifugation.
the analysis returning information about particles secreted from the at least one cell includes one or more of the following alternatives: single-cell omics, live single-cell RNA sequencing, single-cell identification at genotypic and/or phenotypic level, proteomics, mass spectrometry.
the method can be used for identifying cells that can be used for bioprinting purposes, such as for an implant or any other bioprinted construct.
the method can include one or more presteps, wherein single cells, such as at least one cell, are provided, sorted and stored under conditions allowing intracellular particles, such as exosomes, to be secreted from the single cells.
the method can include that the analysis of particles secreted and separated from the at least one cell involves the steps of (i) opening up the particle, to provide access to the RNA content of the particle; (ii) reverse transcribing the RNA content, to convert the RNA content into at least one DNA strand; (iii) amplifying the DNA strand; and (iv) sequencing the DNA strands, to provide genetic information that can be correlated to the individual cell and/or cell population.

The second aspect of this disclosure shows a device for use in the method of the first aspect.

Embodiments of the second aspect include that:
the device is a disposable microfluidic device.
the device is a microchamber for culturing individual cells, which microchamber allows for live cell imaging.
the device allows for extraction and/or analysis of particles secreted from the at least one cells.
the analysis of particles secreted from the at least one cell can be assigned to the individual cell.
the device is implemented on a chip comprising a plurality of microchambers and/or microfluidic devices.

The third aspect of this disclosure shows a system for performing one or more of the steps of the method of the first aspect, thereby enabling, in its broadest applicability, live single cell RNA sequencing.

Embodiments of the third aspect include that the system comprises:
at least one device for use in culturing and analysis of at least one cell, which device comprises a microchamber configured to allow for optical inspection of the at least one cell, and at least one control unit configured to allow for extraction of liquid from the microchamber for analysis, wherein the at least one control unit can be integrated into the device or being a separate unit, for controlling one or more of the steps of the method of the first aspect.
at least one dispensing device, configured to allow for dispensing of particles secreted from the at least one cell into a particle collection well for further analysis of the secreted particles.
at least one of the following components: means for cell sorting, means for multiomics analysis, means for reverse transcription, means for amplification and means for sequencing.

The fourth aspect of this disclosure relates to a computer program which when executed cause at least one processor to carry out the method of the first aspect

The person skilled in the art realizes that the present disclosure is not limited to the preferred embodiments described above. The person skilled in the art further realizes that modifications and variations are possible within the scope of the appended claims.

Additionally, variations to the disclosed embodiments can be understood and effected by the skilled person in practicing the claimed disclosure, from a study of the drawings, the disclosure, and the appended claims.

1. A method of culturing and analyzing at least one cell in a microchamber configured to allow for optical inspection of the at least one cell, wherein liquid is extracted from the microchamber for analysis, wherein the analysis returns information about particles secreted from the at least one cell and that this information can be correlated to the individual cell and/or cell population.

2. The method according to claim 1, wherein the liquid extraction is performed by means of a microfluidic valve and channel or a micropipette.

3. The method according to claim 1, wherein the particles secreted from the at least one cell are extracellular vesicles, such as exosomes.

4. The method according to claim 1, wherein the particles secreted from the at least one cell are separated from the cells by means of a disposable microfluidic device, comprising a microfluidic channel that is thinner than a normal cell, and a particle collection well, wherein the microfluidic channel is used for transporting particles from a cell culturing well to the particle collection well, where the particles can be taken for further analysis.

5. The method according to claim 1, wherein the particles secreted from the at least one cell are separated from the cells by:
(i) adding micromagnetic particles having the ability to bind to the particles secreted from the at least one cell to a cell culturing well;
(ii) aspirating the at least one cell, the particles secreted from the at least one cell, and the magnetic microparticles into an acoustic trap;
(iii) separating the secreted particles, which bind to the magnetic microparticles via physical and/or chemical binding, from the at least one cell; and
(iv) dispensing the secreted particles into a particle collection well, where the particles can be taken for further analysis.

6. The method according to claim 5, wherein in step (iii), the acoustic trap is deactivated, and the magnetic microparticles, having secreted particles bound to them, and the cells are released to at least one source well, in which the magnetic microparticles and the secreted particles are immobilized by applying a magnetic field.

7. The method according to claim 6, wherein the cells are dispensed separately from the at least one source well by applying a positive pressure within the source well.

8. The method according to claim 7, wherein the secreted particles are released from the magnetic microparticles by applying a physical, chemical or enzymatic reaction, and thereafter dispensed from the source well.

9. The method according to claim 1, wherein the particles secreted from the at least one cell are separated from the cells by separating the at least one cell from the particles secreted from the at least one cell using electromechanical principles on a microfluidic chip, so that the secreted particles are collected in a particle collection well, where the particles can be taken for further analysis.

10. The method according to claim 1, wherein the particles secreted from the at least one cell are separated and/or cleaned up by means of lateral displacement, SAW (surface acoustic wave), acoustic technology, coated pillers or beads, electrophoresis or ultracentrifugation.

11. The method according to claim 1, wherein the analysis returning information about particles secreted from the at least one cell includes one or more of the following alternatives: single-cell omics, live single-cell RNA sequencing, single-cell identification at genotypic and/or phenotypic level, proteomics, mass spectrometry.

12. The method according to claim 1, for purposes of identifying cells that can be used for various uses, such as for gene therapy, cell therapy, stem cell therapy, regenerative medicine, bioprinting and/or biofabrication of human tissues and organs for implantation.

13. The method according to claim 1, wherein one or more presteps are performed, wherein single cells, such as at least one cell, are provided, sorted and stored under conditions allowing intracellular particles, such as exosomes, to be secreted from the single cells.

14. The method according to claim 1, wherein the analysis of particles secreted and separated from the at least one cell involves the steps of
(i) opening up the particle, to provide access to the RNA content of the particle;
(ii) reverse transcribing the RNA content, to convert the RNA content into at least one DNA strand;
(iii) amplifying the DNA strand; and
(iv) sequencing the DNA strands, to provide genetic information that can be correlated to the individual cell and/or cell population.

15. A device for use in culturing and analysis of at least one cell, comprising a microchamber configured to allow for optical inspection of the at least one cell, to be used in the method of claim 1.

16. The device according to claim 15, wherein the device is a disposable microfluidic device.

17. The device according to claim 15, wherein the device is a microchamber for culturing individual cells, which microchamber allows for live cell imaging.

18. The device according to claim 15, which allows for extraction and/or analysis of particles secreted from the at least one cell.

19. The device according to claim 18, wherein the analysis of particles secreted from the at least one cell can be assigned to the individual cell and/or cell population.

20. The device according to claim 15, wherein the device is implemented on a chip comprising a plurality of microchambers and/or microfluidic devices.

21. A system comprising at least one device for use in culturing and analysis of at least one cell, which device comprises a microchamber configured to allow for optical inspection of the at least one cell, and at least one control unit configured to allow for extraction of liquid from the microchamber for analysis, wherein the at least one control unit can be integrated into the device or being a separate unit, for controlling one or more of the steps of the method of claim 1.

22. The system according to claim 21, further comprising at least one dispensing device, configured to allow for dispensing of particles secreted from the at least one cell into a particle collection well for further analysis of the secreted particles.

23. The system according to claim 21, further comprising at least one of the following components: means for cell sorting, means for multiomics analysis, means for reverse transcription, means for amplification and means for sequencing.

24. A computer program for use in culturing and analysis of at least one cell in a microchamber, comprising instructions which, when executed on at least one processor, cause the at least one processor to carry out the method of claim 1.
